(19) United States
(12) Patent Application Publication
(10) Pub. No.: US 2015/0305633 A1
(43) Pub. Date: Oct. 29, 2015

(54) Catheter With Inner Surface Pressure Sensor for Providing a Vascular Pressure Measurement for Determining Fractional Flow Reserve

(71) Applicant: Medtronic Vascular Galway, Ballybrit (IE)

(72) Inventors: Gerry McCaffrey, Ballybrit (IE); Fiachra Sweeney, Ballybrit (IE); Barry O'Connell, Ballybrit (IE); Christopher Murphy, Ballybrit (IE); Sean Ward, Ballybrit (IE)

(73) Assignee: Medtronic Vascular Galway, Ballybrit (IE)

(21) Appl. No.: 14/259,896

(22) Filed: Apr. 23, 2014

(51) Int. Cl. A61B 5/0215; A61M 25/09; A61B 5/02

(52) U.S. Cl. CPC A61B 5/02158; A61B 5/02007; A61M 25/09; A61M 2025/0002

(57) ABSTRACT

A catheter includes an elongate shaft including a proximal portion and a distal portion extending from the proximal portion to a distal opening at a distal end of the shaft. The proximal portion defines a proximal guidewire lumen and has a first outer diameter. The distal portion defines a distal guidewire lumen in communication with the proximal guidewire lumen and has a second outer diameter smaller than the first outer diameter. A pressure sensor is coupled to the proximal portion such that the pressure sensor faces the proximal guidewire lumen. When the catheter is tracked to a treatment site within the vasculature, the pressure sensor is disposed proximal the treatment site, the distal opening is disposed distal to the treatment site, and the distal guidewire lumen fills with blood such that the pressure sensor senses a pressure of the blood at the distal end of the shaft.

### FIELD OF THE INVENTION

The invention methods and systems for determining a pressure gradient across a lesion of a vessel for calculating a Fractional Flow Reserve.

### BACKGROUND OF THE INVENTION

The severity of a stenosis or lesion in a blood vessel may be assessed by obtaining proximal and distal pressure measurements relative to the given stenosis and using those measurements for calculating a value of the Fractional Flow Reserve (FFR). FFR is defined as the ratio of a first pressure measurement (P_{d}) taken on the distal side of the lesion and to a second pressure measurement taken on the proximal side of the lesion usually within the aorta (Pa). Conventionally, a sensor placed on the distal portion of a guidewire or FFR wire to obtain the first pressure measurement P_{d}, while an external pressure transducer is fluidly connected via tubing to a guide catheter for obtaining the second or aortic (AO) pressure measurement Pₐ. Calculation of the FFR value provides a lesion specific index of the functional severity of the stenosis in order to determine whether the blockage limits blood flow within the vessel to an extent that treatment is needed. An optimal or normal value of FFR in a healthy vessel is 1.00, while values less than about 0.80 are generally deemed significant and in need of an interventional treatment. Common interventional treatment options include balloon angioplasty and/or stent implantation.

If an interventional treatment is required, the interventional device, such as a balloon catheter, is tracked over a guide wire to the site of the lesion. Conventional FFR wires generally are not desired by clinicians to be used as guide wires for such interventional devices. Accordingly, if an intervention treatment is required, the clinician generally removes the FFR wire, inserts a conventional guide wire, and tracks the interventional device to the treatment site over the conventional guide wire.

Further, some error may be introduced into a blood pressure measurement taken distal of a lesion if a cross-sectional size of the portion of the measurement device that crosses the lesion is too large such that it acts to further restrict blood flow therethrough. Accordingly, there remains a need in the art for devices and methods for obtaining pressure measurements suitable for use in calculating an FFR value for a given stenosis, wherein the clinician may use a conventional guidewire and the cross-sectional size of the portion of the measurement device crossing the lesion is minimized.

### BRIEF SUMMARY OF THE INVENTION

Embodiments hereof relate to a measurement catheter including an elongate shaft including a proximal portion and a distal portion extending from the proximal portion to a distal opening at a distal end of the shaft. The proximal portion defines a proximal guidewire lumen and has a first outer diameter. The distal portion defines a distal guidewire lumen in communication with the proximal guidewire lumen and has a second outer diameter smaller than the first outer diameter. A pressure sensor is coupled to the proximal portion such that the pressure sensor faces the proximal guidewire lumen. When the catheter is tracked to a treatment site within the vasculature, the pressure sensor is disposed proximal the treatment site, the distal opening is disposed distal to the treatment site, and the distal guidewire lumen fills with blood such that the pressure sensor senses a pressure of the blood at the distal end of the shaft. In an embodiment, the distal guidewire lumen is approximately the same size as a guidewire such that the guidewire is retracted when the measurement catheter is in place such that the distal guidewire lumen can be filled with blood. In another embodiment, the distal guidewire lumen is sufficiently larger than the guidewire such that a portion of the distal guidewire lumen adjacent the guidewire can fill with blood and provide an accurate pressure measurement at the pressure sensor while the guidewire is disposed in the distal guidewire lumen.

Embodiments hereof also relate to a measurement catheter including an elongate shaft having a proximal portion and a distal portion extending from the proximal portion to a distal opening at a distal end of the shaft. The proximal portion defines a proximal guidewire lumen and has a first outer diameter. The distal portion defines a distal guidewire lumen in communication with the proximal guidewire lumen and has a second outer diameter smaller than the first outer diameter. A differential pressure sensor is coupled to the proximal portion such that the differential pressure sensor includes an inner portion facing the proximal guidewire lumen and an outer portion facing outside of an outer surface of the elongate shaft. The proximal guidewire lumen and distal guidewire lumen are configured to receive a guidewire and to provide fluid communication between the differential pressure sensor and the distal opening. When the catheter is tracked to a treatment site within the vasculature, the differential pressure sensor is disposed proximal the treatment site, the distal opening is disposed distal to the treatment site, and the distal guidewire lumen fills with blood such that the differential pressure sensor senses a pressure difference between blood flowing distally past the outer portion of the differential pressure sensor and blood at the distal end of the shaft sensed by the inner portion of the differential blood pressure via the blood filling the distal guidewire lumen. In an embodiment, the distal guidewire lumen is approximately the same size as a guidewire such that the guidewire is retracted when the measurement catheter is in place such that the distal guidewire lumen can be filled with blood. In another embodiment, the distal guidewire lumen is sufficiently larger than the guidewire such that a portion of the distal guidewire lumen adjacent the guidewire can fill with blood and provide an accurate differential pressure measurement at the differential pressure sensor while the guidewire is disposed in the distal guidewire lumen.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is an illustration of a system for measuring FFR with a distal portion thereof shown within a vessel including a lesion, the system including a measurement catheter including a pressure sensor facing a guidewire lumen thereof and a guidewire, in accordance with an embodiment hereof

FIG. 2 is an illustration of the catheter of claim FIG. 1 in partial longitudinal cross-section.

FIG. 3 is a cross-sectional view of the catheter taken along line 3-3 of FIG. 2.

FIG. 4 is a cross-sectional view of the catheter taken along line 4-4 of FIG. 2.

FIG. 5 is a detailed view of a portion of the catheter of FIG. 1 including a pocket and the pressure sensor.

FIG. 6 is a detailed view of another embodiment of the portion of the catheter of FIG. 1 including the pocket and the pressure sensor.

FIG. 6A is a detailed view of FIG. 6.

FIGS. 7-10 are schematic diagrams of an embodiment of a method for measuring FFR using the system of FIG. 1.

FIG. 11 is a detail view of a portion of the catheter of FIG. 1 including seals.

FIG. 12 is an illustration of a system for measuring FFR, the system including a measurement catheter including a pressure sensor facing a guidewire lumen thereof and a guidewire, in accordance with another embodiment hereof.

FIG. 13 is an illustration of a distal portion of the system of FIG. 12.

FIG. 14 is an illustration of a distal portion of the system of FIG. 12 disposed in a vessel illustrating a method of measuring FFR using the system of FIG. 12.

FIG. 15 is an illustration of a system for measuring FFR, the system including a measurement catheter including a differential pressure sensor and a guidewire, in accordance with another embodiment hereof

FIG. 16 is an embodiment of a cross sectional view of the portion of the system of FIG. 15 at the differential pressure sensor.

FIG. 17 is an illustration of a distal portion of the system of FIG. 15 disposed in a vessel illustrating a method of measuring FFR using the system of FIG. 15.

FIG. 18 is an illustration of a portion of a measurement catheter for measuring FFR, the measurement catheter including first pressure sensor and a second pressure sensor, in accordance with another embodiment hereof

FIG. 19 is an illustration of a distal portion of the measurement catheter of FIG. 18 disposed in a vessel illustrating a method of measuring FFR using the measurement catheter of FIG. 15.

FIG. 20 is an illustration of the measurement of FIG. 18 with a different arrangement of the first and second pressure sensors.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” and “distally” are positions distant from or in a direction away from the clinician. “Proximal” and “proximally” are positions near or in a direction toward the clinician.

The following detailed description is merely exemplary in nature and is not intended to limit the invention or the application and uses of the invention. Although the description of the invention is in the context of treatment of blood vessels such as the coronary arteries, the invention may also be used in any other body passageways where it is deemed useful such as but not limited to peripheral arteries, carotid arteries, renal arteries, and/or venous applications. Furthermore, there is no intention to be bound by any expressed or implied theory presented in the preceding technical field, background, brief summary or the following detailed description.

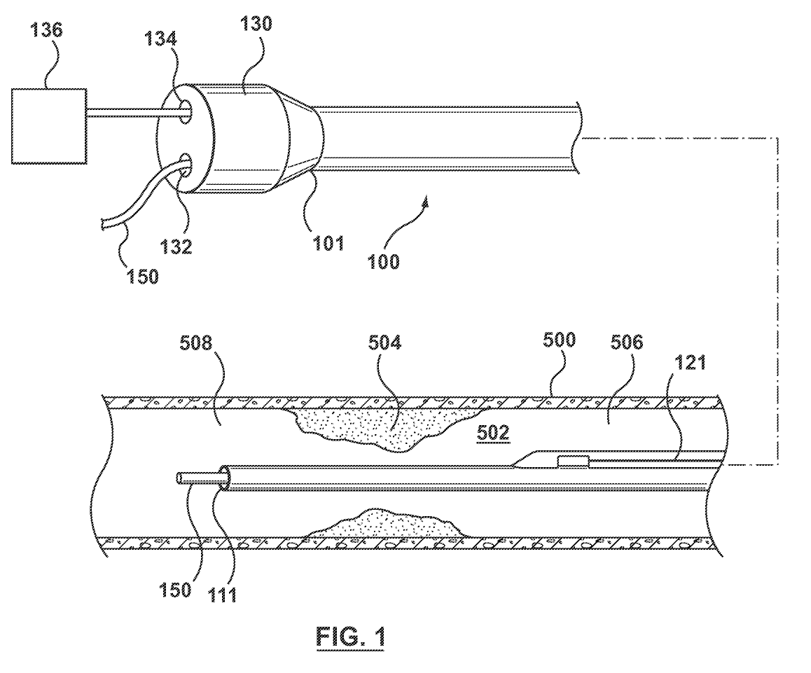

With reference to FIG. 1, a measurement catheter 100 is shown with a proximal portion thereof extending outside of a patient and a distal portion thereof positioned in situ within a lumen 502 of a patient vessel 500 having a stenosis or lesion 504. In an embodiment hereof, the vessel 500 is a blood vessel such as but not limited to a coronary artery. Lesion 504 is generally representative of any blockage or other structural arrangement that results in a restriction to the flow of fluid through lumen 502 of vessel 500. Lesion 504 may be a result of plaque buildup, including without limitation plaque components such as fibrous, fibro-lipidic (fibro fatty), necrotic core, calcified (dense calcium), blood, fresh thrombus, and mature thrombus. Generally, the composition of lesion will depend on the type of vessel being evaluated. In that regard, it is understood that embodiments hereof are applicable to various types of blockage or other narrowing of a vessel that results in decreased fluid flow.

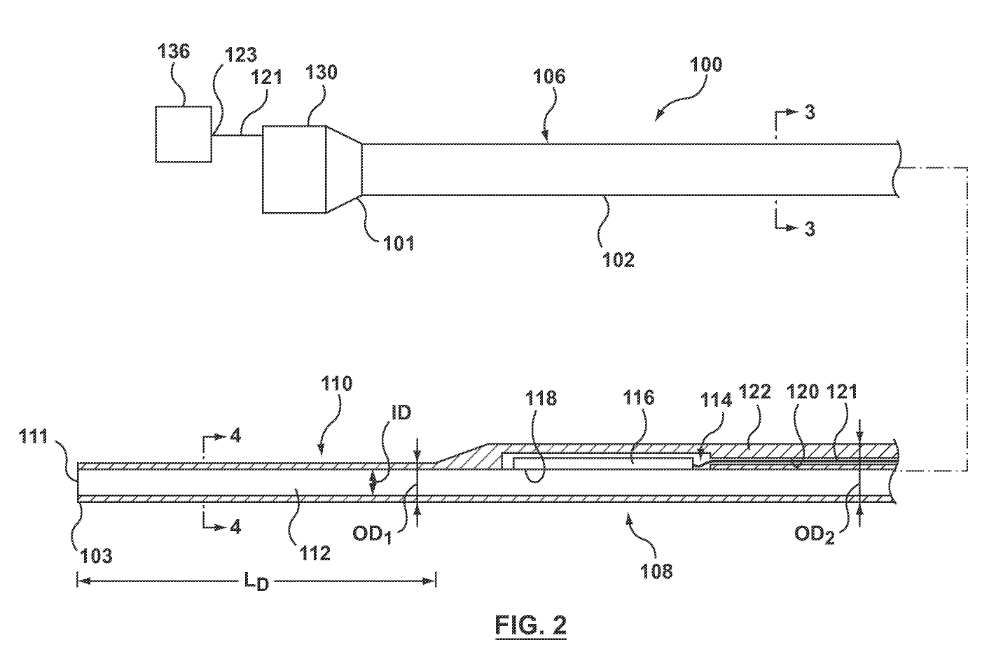

Measurement catheter 100 is shown in FIG. 2 with a distal portion thereof in partial cut-away. Measurement catheter 100 includes an elongate shaft 102 having a proximal end 101 coupled to a handle or luer 130 and a distal end 103 having a distal opening 111. Elongate shaft 102 further includes a proximal portion 106, an intermediate or pressure sensing portion 108, and a distal portion 110, as described in more detail below. Elongate shaft 102 may be formed of a polymeric material, non-exhaustive examples of which include polyethylene, PEBA, polyamide and/or combinations thereof, either blended or co-extruded. Optionally, the catheter shaft or some portion thereof may be formed as a composite having a reinforcement material incorporated within a polymeric body in order to enhance strength and/or flexibility. Suitable reinforcement layers include braiding, wire mesh layers, embedded axial wires, embedded helical or circumferential wires, and the like. In one embodiment, for example, at least a proximal portion of elongate shaft 102 may be formed from a reinforced polymeric tube.

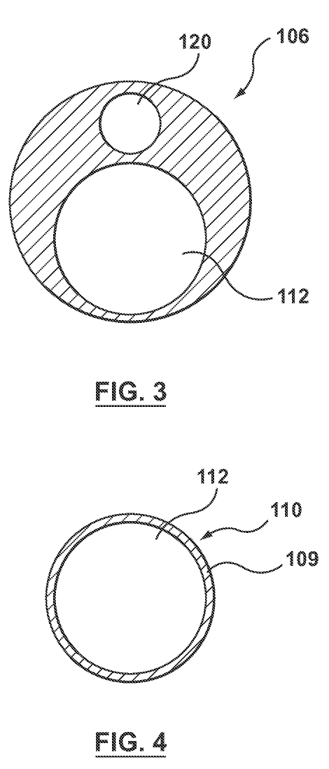

Elongate shaft 102 includes a guide wire lumen 112 extending therethrough. In the embodiment of FIGS. 1-10, guide wire lumen 112 extends through proximal portion 106, pressure sensing portion 108, and distal portion 110. However, instead of the over-the-wire configuration shown in FIGS. 1-11, catheter 100 may have a rapid exchange configuration wherein guide wire lumen 112 extends through distal portion 110 and intermediate portion 108, and the guidewire exits shaft 102 through a rapid exchange port (not shown) in proximal portion 106, as would be understood by those skilled in the art. With additional reference to the cross-sectional view of FIG. 3, proximal portion 106 of elongated shaft 102 in one embodiment defines two separate lumens, a proximal portion of guide wire lumen 112 and a second or pressure sensor wire lumen 120, extending parallel or side-by-side to each other along proximal portion 106. Although depicted as circular in cross-section, one or more lumen(s) of elongated shaft 102 may have any suitable cross-section including for example circular, elliptical, or crescent-shaped. As explained in more detail below, pressure sensing wire lumen 120 extends to pressure sensing portion 108 of elongate shaft 102 to be coupled to a pressure sensor 116. As further explained below, pressure sensor wire lumen 120 may be eliminated in embodiments wherein a signal from pressure sensor 116 is sent to a computing device 136 other than via a wire 121 in a dedicated pressure sensor wire lumen 120, such as, but not limited to, wireless transmission or integration of wire 121 into the wall of elongate shaft 102.

Distal portion 110 of elongate shaft 102 is configured to receive a guidewire 150 in a distal portion of guidewire lumen 112 thereof. Further, as shown in FIGS. 1 and 8-10, distal portion 110 is sized to extend from a proximal side 506 of lesion 504, through lesion 504, and to a distal side 508 of lesion 504 such that distal opening 111 is disposed on distal side 508 of lesion 504. Accordingly, in an embodiment, distal portion 110 has a length L_{D} in the range of 25-150 mm. However, length L_{D} may be any length suitable such that distal portion 110 may extend from proximal side 506 to distal side 508. Further, because distal portion 110 is configured to extend through lesion 504, the cross-sectional dimension or profile of distal portion 110 is minimized such as not to disrupt blood flow through lesion 504 in order to obtain an accurate FFR measurement, as explained in more detail below. FIG. 4 shows a cross-section of distal portion 110 of elongate shaft 102 taken along line 4-4 of FIG. 2. As can be seen in FIG. 4, distal portion 110 includes a shaft wall 109 defining a portion of lumen 112 extending through distal portion 110. Thus, because distal portion 110 receives guidewire 150 therein, the distal portion of guide wire lumen 112 disposed in distal portion 110 is sized to receive guide wire 150. However, in the embodiment of FIGS. 1-6, any additional cross-sectional size or profile of distal portion 110 is minimized. Accordingly, in an embodiment to be used with a 0.014 inch guidewire 150, distal portion 110 has an inner diameter ID of approximately 0.0155 inch and an outer diameter OD₁ of approximately 0.018 inch. However, these sizes are provided only as examples and may change depending on various factors such as, but not limited to, the size of the guidewire, the size of the pressure sensor, the particular vessel, and various other factors.

Pressure sensing portion 108 of elongate shaft 102 is located proximally of distal portion 110 and distal of proximal portion 106. Pressure sensing portion 108, as shown in FIG. 2, includes guidewire lumen 112 and a pressure sensor 116. Pressure sensor 116 includes a pressure sensing surface 118 that faces guide wire lumen 112 such that pressure sensing surface 118 measures a pressure of a fluid within guidewire lumen 112, as explained in more detail below. In the embodiment shown in FIG. 2, pressure sensor 116 is disposed in a pocket 114 of a thickened portion 122 of elongated shaft 102. Pressure sensor 116 may be a piezo-resistive pressure sensor, a piezo-electric pressure sensor, a capacitive pressure sensor, an electromagnetic pressure sensor, an optical pressure sensor, and/or combinations thereof. In one non-limiting example pressure sensor 116 is about 330 microns by 180 microns by 1100 microns in size. However, other sized pressure sensors may be used. As shown in FIG. 2, thickened portion 122 needs to accommodate pressure sensor 116. Accordingly, thickened portion 122 of elongate shaft 102 has an outer diameter OD₂ which is larger than the outer diameter OD₁ of distal portion 110 of elongate shaft 102. However, depending on the size of pressure sensor 116, the OD₁ and OD₂ of the elongate shaft 102 could have the substantially the same diameter. In one embodiment, outer diameter OD₂ of thickened portion is in the range of 0.018 inch-0.036 inch in order to accommodate pressure sensor 116. However, OD₂ may vary depending on the size of pressure sensor 116, thickness of elongate shaft 102, and other factors used to determine the diameter or profile of shafts.

In an embodiment, pocket 114 is in communication with pressure sensor wire lumen 120 such that any communication wire(s) 121 from pressure sensor 116 may extend from pocket 114 proximally through pressure sensor wire lumen 120, through a corresponding lumen in luer 130 exiting through proximal port 134 to a computing device 136 coupled to proximal end 123 of communication wire 121. Proximal end 123 of communication wire 121 may be coupled to computing device 136 via various communication pathways, including but not limited to one or more physical connections including electrical, optical, and/or fluid connections, a wireless connection, and/or combinations thereof. Accordingly, it is understood that additional components (e.g., cables, connectors, antennas, routers, switches, etc.) not illustrated in FIG. 1 may be included to facilitate communication between the proximal end 123 of communication wire 121 and computing device 136. In other embodiments, instead of a dedicated pressure sensor wire lumen 120, communication between pressure sensor 116 and computing device 136 may be accomplished wirelessly, or communication wires 121 may be incorporated into the wall of elongate shaft 102.

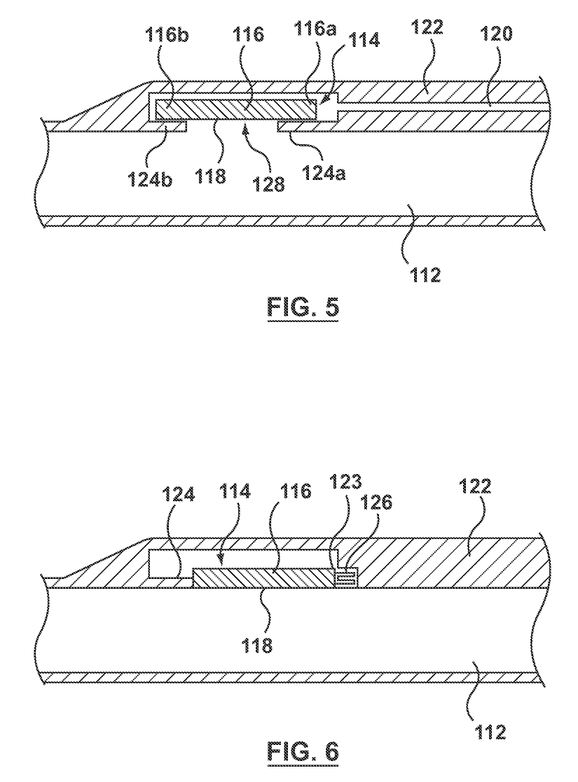

FIG. 5 shows an embodiment of pressure sensor 116 disposed in pocket 114 in pressure sensing portion 108 of shaft 103. In particular, a portion of thickened portion 122 is reduced to form pocket 114 with arms 124a/124b extending from ends of pocket 114 on the guidewire lumen side of pocket 114. Arms 124a/124b do not extend all the way to each other such that a gap 128 is formed between arms 124a/124b, as shown in FIG. 5. Pressure sensor is disposed in pocket 114 such that ends 116a/116b of pressure sensor 116 overlap corresponding arms 124a/124b and pressure sensing surface 118 is disposed adjacent gap 128, as shown in FIG. 5. Ends 116a/116b of pressure sensor 116 may be coupled to arms 124a/124b using techniques such as, but not limited to, adhesives, mechanical connections, and other similar coupling mechanisms.

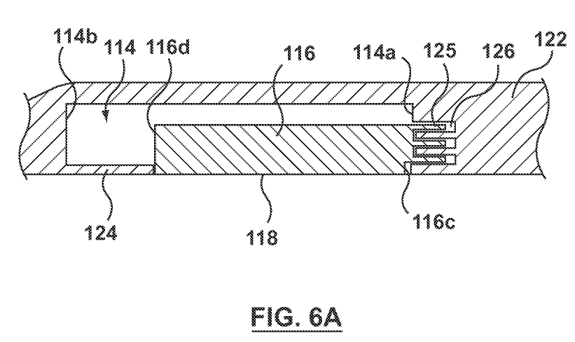

In another embodiment, shown in FIGS. 6 and 6A, a first end 116c of pressure sensor 116 includes tabs 125 which fit into slots 126 in a first end 114a of pocket 114. A second end 116d of pressure sensor 116 abuts an arm 124 extending from a second end 114b of pocket 114. In the embodiment shown in FIG. 6, second end 116d of pressure sensor 116 abuts arm 124. However, the second end 116d of pressure sensor 116 may overlap arm 124 similar to the embodiment shown in FIG. 5. Further, there are numerous other ways to couple pressure sensor 116 to thickened portion 122 of shaft 102 that are equally suitable provided that pressure sensing surface 118 of pressure sensor 116 is exposed to fluid filling guidewire lumen 112, as described below.

Although proximal portion 106, pressure sensing portion 108, and distal portion 110 of elongate shaft have been described separately, they are described in such a manner for convenience and elongate shaft 102 may be constructed unitarily such that the portions described are part of a unitary shaft. However, different portions of elongate shaft 102 may also be constructed separately and joined together.

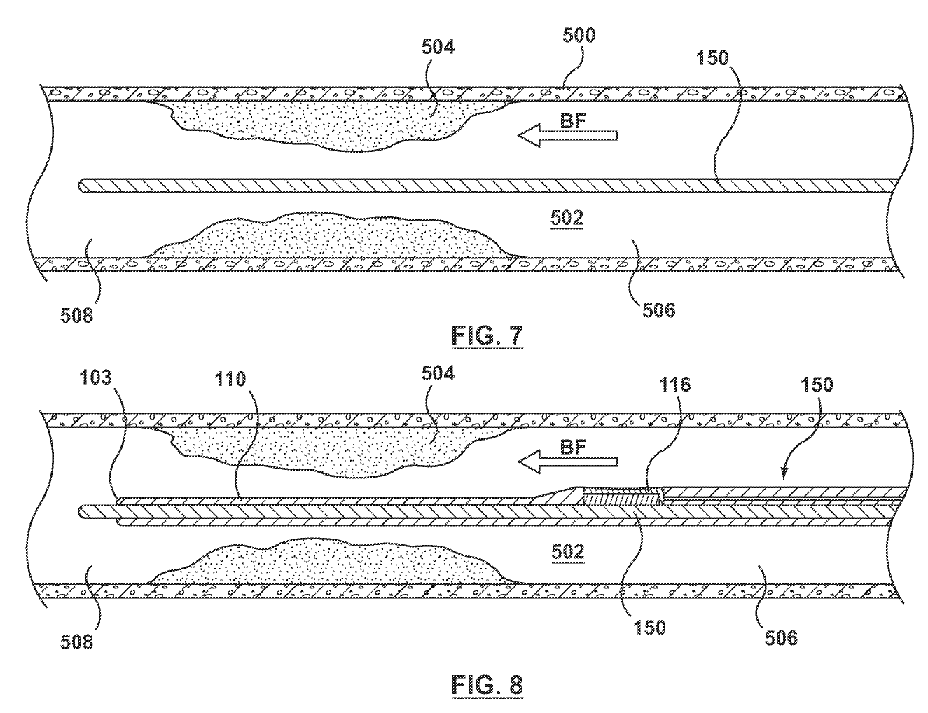

A method of measuring FFR using measurement catheter 100 will now be described with reference to FIGS. 7-11. As would be understood by those skilled in the art, when measuring FFR a guide catheter (not shown) is advanced through the vasculature such that the guide catheter is disposed within the aorta with a distal end thereof disposed within the aorta at an ostium of the aorta adjacent the branch vessel 500 within which lesion 504 is located. As shown in FIG. 7, guidewire 150 is advanced intraluminally through the guide catheter, into vessel 500 within lumen 502 to the site of lesion 504. In the embodiment shown, guidewire 150 is advanced from proximal side 506 of lesion 504 to distal side 508 of lesion 504, which is also consistent with the direction of the blood flow BF, as indicated by the arrow BF in FIG. 7. In an embodiment, vessel 500 is a coronary artery, but vessel 500 may be other vessels in which it may be desirable to measure pressure, and in particular, to measure FFR.

Thereafter, as shown in FIG. 8, measurement catheter 100 is tracked or advanced over indwelling guidewire 150 to the target site such that distal end 103 of elongate shaft 102 is positioned distal of lesion 504. As can be seen in FIG. 8, pressure sensor 116 is disposed proximal of lesion 504 such that the smaller profile distal portion 110 of elongate shaft 102 is disposed through lesion 504.

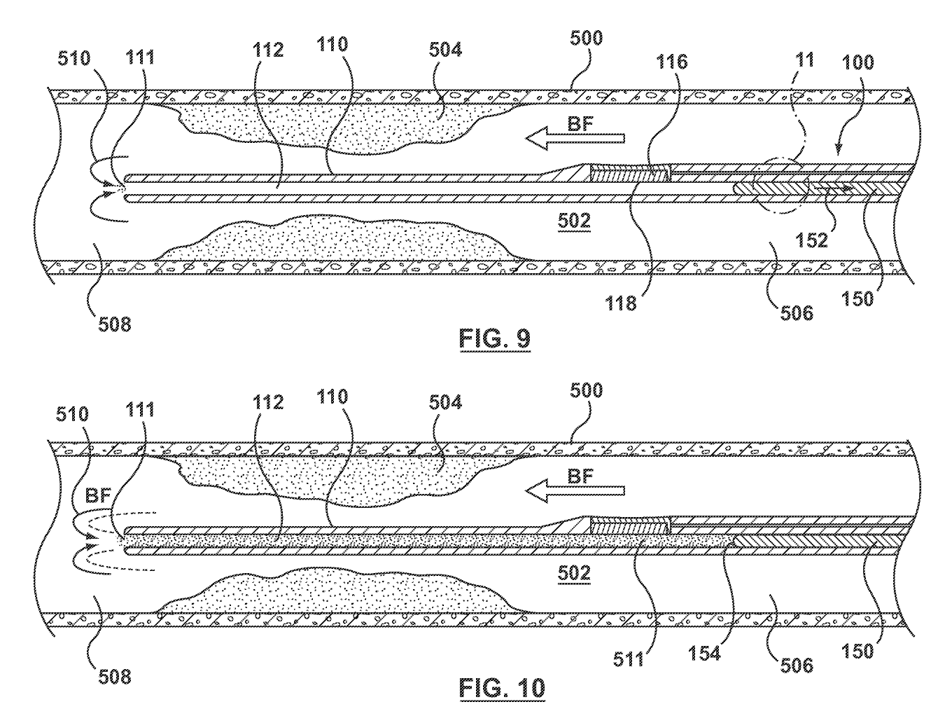

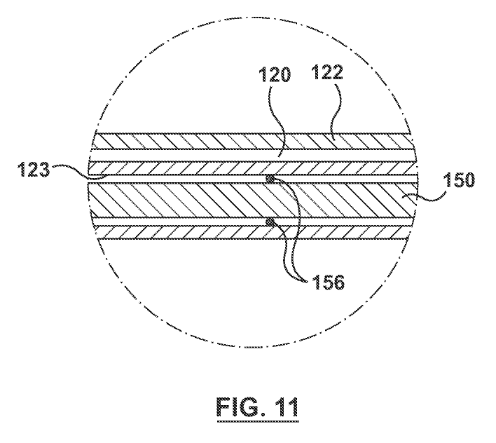

With measurement catheter 100 in place, guidewire 150 is retracted proximally until guidewire 150 is disposed proximal of pressure sensor 116, as shown in FIG. 9 and indicated by arrow 152. With guidewire 150 proximally retracted, blood 511 may enter distal opening 111 as indicated by arrows 510 and fill guidewire lumen 112 of elongate shaft 102 from a location at a distal end 154 of guidewire 150 to distal opening 111, as shown in FIGS. 9 and 10. Further, as shown in FIG. 11, a seal 156 may be provided along an inner surface 123 of thickened portion 122 of elongated shaft 102 at approximately the location to where guidewire 150 is retracted such that blood will not fill guidewire lumen 112 proximal of seal 156. Although seal 156 is shown in thickened portion 122, seal 156 may be provided anywhere proximal of pressure sensor 116. Seal 156 may be any seal that can prevent flow of blood between an inner surface of a shaft and an outer surface of a guidewire, such as, but not limited to a localized reduction in inner diameter, hemostatic seals, 0-rings, duckbill or diaphragm silicone, soft elastomer seals, and other various.

With guidewire lumen 112 filled with blood 511, pressure sensing surface 118 of pressure sensor 116 measures the pressure of blood 511 within guidewire lumen 112. The pressure measured by pressure sensor 116 is representative of the pressure of blood 511 at the distal side 508 of lesion 504 where the blood 511 enters distal opening 111 because the blood filing lumen 112 transmits the pressure to the sensor. Accordingly, the pressure measured by pressure sensor 116 is the distal pressure measurement, or P_{d}, used in calculating FFR. In one embodiment, adenosine is administered either intracoronary at the site, bolus, or intravenously by continuous infusion for providing an accurate distal pressure measurement (P_{d}) for an FFR value. A proximal pressure measurement Pₐ, which is taken in the aorta by an external AO pressure transducer associated with the guide catheter, and a simultaneous pressure measurement P_{d} taken with pressure sensor 116 of measurement catheter 100 are then obtained to provide the FFR value, i.e., P_{d}/Pₐ, for the lesion. The proximal pressure measurement Pₐ and distal pressure measurement P_{d} are communicated to computing device 136. Computing device 136 may include such components as a CPU, a display device, an amplification and filtering device, an analog-to-digital converter, and various other components. Computing device 136 receives the proximal pressure measurement Pₐ and distal pressure measurement P_{d}, processes them as known by those skilled in the art, and provides a continuous display of FFR measurement.

When the FFR measurement is completed, measurement catheter 100 may then be completely withdrawn from the patient or repositioned in vivo at another lesion and the process repeated.

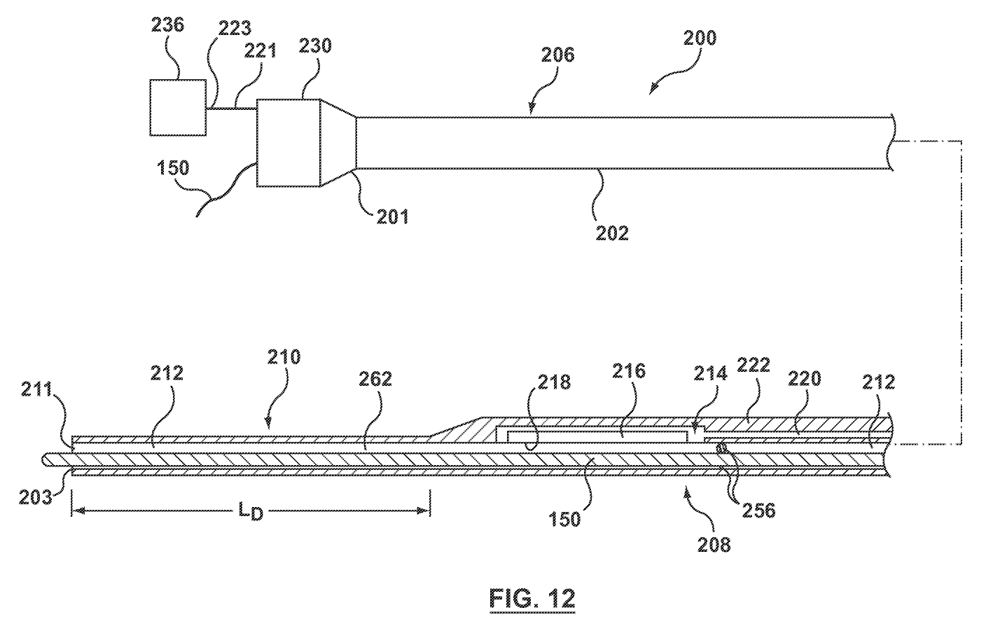

FIGS. 12-14 show a measurement catheter 200 and method of using measurement catheter 200 to obtain a distal pressure measurement P_{d} in accordance with another embodiment hereof. Measurement catheter 200 is similar to measurement catheter 100 except that guidewire 150 does not need to be retracted for pressure sensor 216 to obtain the distal pressure measurement P_{d}, as described in more detail below.

Measurement catheter 200 includes an elongate shaft 202 having a proximal end 201 coupled to a handle or luer 230 and a distal end 203 having a distal opening 211. Elongate shaft 202 includes a proximal portion 206, an intermediate or pressure sensing portion 208, and a distal portion 210, as described in more detail below. Elongate shaft 202 may be formed of the materials described above with respect to elongate shaft 102.

Elongate shaft 202 includes a guide wire lumen 212 extending therethrough. In the embodiment shown in FIGS. 12-14, guide wire lumen 212 extends through proximal portion 206, pressure sensing portion 208, and distal portion 210. However, those skilled in the art would recognize that instead of the over-the-wire configuration shown in FIGS. 10-12, catheter 200 may have a rapid exchange configuration wherein guide wire lumen 212 extends through distal portion 210 and intermediate portion 208, and guidewire 150 exits shaft 202 through a rapid exchange port (not shown) in proximal portion 206, as would be understood by those skilled in the art. Proximal portion 206 may be similar to proximal portion 106 of catheter 100 shown in FIG. 3, including guidewire lumen 212 and pressure sensor wire lumen 220 extending therethrough. Further, although depicted as circular in cross-section, one or more lumen(s) of elongated shaft 202 may have any suitable cross-section including for example circular, elliptical, or crescent-shaped. Pressure sensing wire lumen 220 extends to pressure sensing portion 208 of elongate shaft 202 to be coupled to pressure sensor 216. As further explained below, pressure sensor wire lumen 220 may be eliminated in embodiments wherein a signal from pressure sensor 216 is sent to computing device 136 other than via a wire 221 in a dedicated pressure sensor wire lumen 220, such as, but not limited to, wireless transmission or integration of wire 221 into the wall of elongate shaft 202.

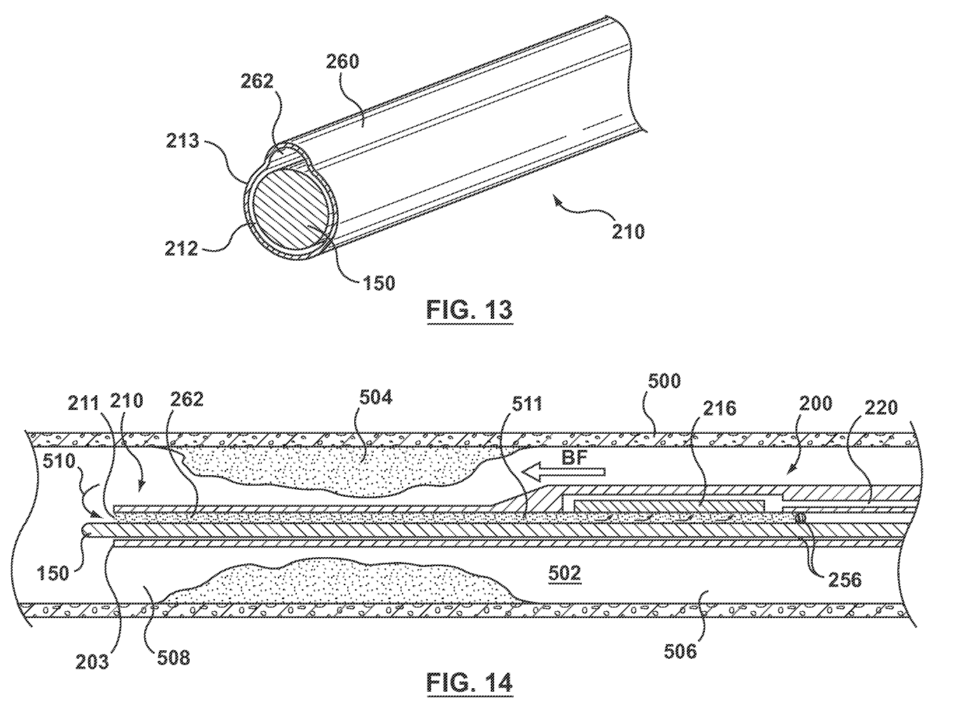

Distal portion 210 of elongate shaft 202 is configured to receive a guidewire 150 in guide wire lumen 212. However, as distinguished from distal portion 110 of catheter 100, guidewire lumen 212 in distal portion 210 is sufficiently larger than guidewire 150 such that blood can fill a portion 262 of guidewire lumen 212 that is not occupied by guidewire 150, as shown, for example, in FIGS. 12-14. As shown in FIG. 13, an extension or bump 260 extends longitudinally along substantially the length of the distal portion 210 of shaft 202 from distal opening 211 to form the added portion 262 of guidewire lumen 212. In one embodiment, the bump 260 extends from distal opening 211 into the pressure sensing portion 208 of shaft 202 to be in fluid communication with the pocket 214. In the embodiment shown in FIGS. 12-14, bump 260 gives distal portion 210 a pear-shaped appearance. However, other shapes may be used provided that portion 262 is sufficiently large to fill with blood and provide an accurate measurement by pressure sensor 216. In one embodiment, portion 262 is has a height H in the range of approximately 0.002 inch-0.010 inch and a width W in the range of approximately 0.002 inch-0.010 inch. However, other sizes may be utilized provided there is sufficient room around guidewire 150 for sufficient blood to filled portion 262 such that pressure sensor 216 can obtain an accurate pressure measurement, as described in detail below.

Further, as shown in FIG. 14, distal portion 210 is sized to extend from a proximal side 506 of lesion 504, through lesion 504, to a distal side 508 of lesion 504 such that distal opening 211 is disposed on distal side 508 of lesion 504. Accordingly, in an embodiment, distal portion 210 has a length L_{D} in the range of 25-150 mm. (However, length L_{D} may be any length suitable such that distal portion 210 may extend from proximal side 506 to distal side 508. Further, because distal portion 210 is configured to extend through lesion 504, the cross-sectional dimension or profile of distal portion 210 is minimized such as not to disrupt blood flow through lesion 504 in order to obtain an accurate FFR measurement, as explained in more detail below. Thus, because distal portion 210 receives guide wire 150 therein, guidewire lumen 212 of distal portion 210 is sized to receive guide wire 150 and portion 262, as explained above.

Pressure sensing portion 208 of elongate shaft 202 is located proximally of distal portion 210 and distal of proximal portion 206. Pressure sensing portion 208, as shown in FIG. 12, includes guide wire lumen 212 and a pressure sensor 216. Pressure sensor 216 includes a pressure sensing surface 218 that faces guide wire lumen 212 such that pressure sensing surface 218 measures a pressure of a fluid within guide wire lumen 212, as explained in more detail below. In the embodiment shown in FIG. 12, pressure sensor 216 is disposed in a pocket 214 of a thickened portion 222 of elongate shaft 202. Pressure sensor 216 may be a piezo-resistive pressure sensor, a piezo-electric pressure sensor, a capacitive pressure sensor, an electromagnetic pressure sensor, an optical pressure sensor, and/or combinations thereof, or other pressure sensors suitable for measuring blood pressure. As shown in FIG. 12, thickened portion 222 needs to accommodate pressure sensor 216. Pressure sensor 216 may be disposed in pocket 214 as described with respect to FIGS. 5 and 6 above or other configurations to accommodate pressure sensor 216.

In an embodiment, pocket 214 is in communication with pressure sensor wire lumen 220 such that any communication wire(s) 221 from pressure sensor 216 may extend from pocket 214 proximally through pressure sensor wire lumen 220, through a corresponding lumen in luer 230 exiting through proximal port (not shown) to computing device 236 coupled to proximal end 223 of communication wire 221. Proximal end 223 of communication wire 221 may be coupled to computing device 136 via various communication pathways, including but not limited to one or more physical connections including electrical, optical, and/or fluid connections, a wireless connection, and/or combinations thereof. Accordingly, it is understood that additional components (e.g., cables, connectors, antennas, routers, switches, etc.) not illustrated in FIG. 12 may be included to facilitate communication between the proximal end 223 of communication wire 221 and computing device 136. In other embodiments, instead of a dedicated pressure sensor wire lumen 220, communication between pressure sensor 216 and computing device 136 may be accomplished wirelessly, or communication wires 221 may be incorporated into the wall of elongate shaft 202, or communication may be accomplished in various other ways.

Utilizing catheter 200 instead of catheter 100 allows the distal pressure measurement P_{d} to be taken without retracting guidewire 150, as compared to the method described with respect to catheter 100 as shown in FIG. 9. Elimination of the retraction of guidewire 150 may be desirable because clinicians often do not favor moving the guidewire once it is in the desired position/location. The tradeoff in this embodiment is a slightly larger profile crossing the lesion 504.

Thus, in a method for measuring FFR using measurement catheter 200, a guide catheter (not shown) is advanced through the vasculature such that the guide catheter is disposed within the aorta with a distal end of the guide catheter disposed within the aorta at an ostium of the aorta adjacent the branch vessel 500 within which lesion 504 is located. Guidewire 150 is advanced intraluminally through the guide catheter, into vessel 500 within lumen 502 to the site of lesion 504, as shown and described with respect to FIG. 7. In the embodiment shown, guidewire 150 is advanced from proximal side 506 of lesion 504 to distal side 508 of lesion 504, which is also consistent with the direction of the blood flow BF, as indicated by the arrow BF in FIG. 7. In an embodiment, vessel 500 is a coronary artery, but vessel 500 may be other vessels in which it may be desirable to measure pressure, and in particular, to measure FFR.

Thereafter, measurement catheter 200 is tracked or advanced over indwelling guidewire 150 to the target site such that distal end 203 of elongate shaft 202 is positioned distal of lesion 504. Similar to the embodiment shown in FIG. 8, and as shown in FIG. 14, pressure sensor 216 is disposed proximal of lesion 504 such that the smaller profile distal portion 210 of elongate shaft 202 is disposed through lesion 504 to distal side 508 of lesion 504.

With measurement catheter 200 in place, blood 511 may enter distal opening 211 as indicated by arrow 510 in FIG. 14. Blood 511 entering distal opening 211 fills portion 262 of guidewire lumen 212 of distal portion 210 not occupied by guidewire 150, as also shown in FIG. 14. Thus, blood 511 fills portion 262 of lumen 212 from distal opening 211 to a location just proximal of pressure sensor 216 where seal 256 prevents blood 511 from filling lumen 212 proximal to seal 256. With portion 262 of guidewire lumen 212 filled with blood 511, pressure sensing surface 218 of pressure sensor 216 measures the pressure of blood 511 within portion 262 of guidewire lumen 212. The pressure measured by pressure sensor 216 is representative of the pressure of blood 511 at the distal side 508 of lesion 504 where the blood 511 enters distal opening 211. Accordingly, the pressure measured by pressure sensor 216 is the distal pressure measurement, or P_{d}, used in calculating FFR. In one embodiment, adenosine is administered either intracoronary at the site, bolus, or intravenously by continuous infusion for providing an accurate distal pressure measurement (P_{d}) for an FFR value. A proximal pressure measurement Pₐ, which is taken in the aorta by an external AO pressure transducer associated with the guide catheter, and a simultaneous pressure measurement P_{d} taken with pressure sensor 216 of measurement catheter 200 are then obtained to provide the FFR value, i.e., P_{d}/Pₐ, for the lesion. The proximal pressure measurement Pₐ and distal pressure measurement P_{d} are communicated to computing device 136. Computing device 136 may include such components as a CPU, a display device, an amplification and filtering device, an analog-to-digital converter, and various other components. Computing device 136 receives the proximal pressure measurement Pₐ and distal pressure measurement P_{d}, processes them as known by those skilled in the art, and provides a continuous display of FFR measurement.

When the FFR measurement is completed, measurement catheter 100 may then be completely withdrawn from the patient or repositioned in vivo at another lesion and the process repeated.

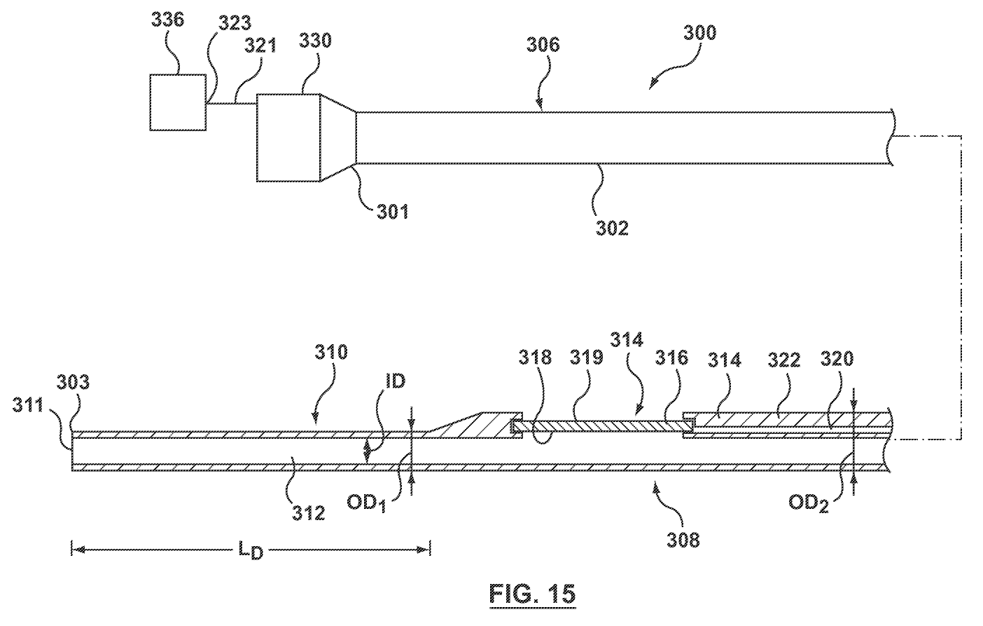

FIGS. 15-17 show a measurement catheter 300 and method of using measurement catheter 300 to obtain a distal differential pressure measurement ΔP in order to calculate FFR in accordance with another embodiment hereof. Measurement catheter 300 is similar to measurement catheter 100 except that pressure sensor 316 measures a differential pressure between an outside facing surface and an inside facing surface, as explained in more detail below. Further, while measurement catheter 300 is described below as similar to measurement catheter 100 in that an inner diameter ID of the distal portion 310 of measurement catheter 300 is sized approximately the same size as guidewire 150, distal portion 310 may instead be sized similar to distal portion 210 of measurement catheter 200 such that guidewire 150 need not be retracted.

Measurement catheter 300 includes an elongate shaft 302 having a proximal end 301 coupled to a handle or luer 330 and a distal end 303 having a distal opening 311. Elongate shaft 302 includes a proximal portion 306, an intermediate or pressure sensing portion 308, and a distal portion 310, as described in more detail below. Elongate shaft 302 may be formed of the materials described above with respect to elongate shaft 102.

Elongate shaft 302 includes a guide wire lumen 312 extending therethrough. In the embodiment shown in FIGS. 15-17, guide wire lumen 312 extends through proximal portion 306, pressure sensing portion 308, and distal portion 310. However, instead of the over-the-wire configuration shown in FIGS. 15-17, catheter 300 may have a rapid exchange configuration wherein guide wire lumen 312 extends through distal portion 310 and intermediate portion 308, and guidewire 150 exits shaft 302 through a rapid exchange port (not shown) in proximal portion 306, as would be understood by those skilled in the art. Proximal portion 306 may be similar to proximal portion 106 of catheter 100 shown in FIG. 3, including guidewire lumen 312 and pressure sensor wire lumen 320 extending therethrough. Further, although depicted as circular in cross-section, one or more lumen(s) of elongate shaft 302 may have any suitable cross-section including for example circular, elliptical, or crescent-shaped. Pressure sensing wire lumen 320 extends to pressure sensing portion 308 of elongate shaft 302 to be coupled to differential pressure sensor 316. As further explained below, pressure sensor wire lumen 320 may be eliminated in embodiments wherein a signal from differential pressure sensor 316 is sent to computing device 336 other than via a wire 321 in a dedicated pressure sensor wire lumen 320, such as, but not limited to, wireless transmission or integration of wire 321 into the wall of elongate shaft 302.

Distal portion 310 of elongate shaft 302 is configured to receive a guidewire 150 in guide wire lumen 312. As explained above with respect to the embodiment of FIGS. 1-10, in the embodiment of FIGS. 15-17, guidewire lumen 312 in distal portion 310 of shaft 302 is sized to receive guidewire 150. However, any additional cross-sectional size or profile of distal portion 310 is minimized such as to minimize the profile of distal portion 310 crossing lesion 504. Accordingly, in an embodiment for use with a 0.014 inch guidewire, distal portion 310 has an inner diameter ID of approximately 0.0155 inch and an outer diameter OD₁ of approximately 0.018 inch. However, these sizes are provided only as examples and may change depending on various factors such as, but not limited to, the size of the guidewire, the size of the pressure sensor, the particular vessel, and various other factors. Further, if the profile of FIGS. 15-17 is used (similar to FIGS. 1-11), guidewire 150 will need to be retracted to filled lumen 312 such that the differential pressure measurement described below may be taken. However, instead of the profile of FIGS. 1-11, distal portion 310 of measurement catheter 300 may instead have the profile of the embodiment of FIGS. 12-14 such that the guidewire 150 need not be retracted for lumen 312 to have sufficient fluid for a differential pressure measurement at differential pressure sensor 316.

Further, as shown in FIG. 17, distal portion 310 is sized to extend from a proximal side 506 of lesion 504, through lesion 504, and to a distal side 508 of lesion 504 such that distal opening 311 is disposed on distal side 508 of lesion 504. Accordingly, in an embodiment, distal portion 310 has a length L_{D} in the range of 25-150 mm. However, length L_{D} may be any length suitable such that distal portion 310 may extend from proximal side 506 to distal side 508. Further, because distal portion 310 is configured to extend through lesion 504, the cross-sectional dimension or profile of distal portion 310 is minimized such as not to disrupt blood flow through lesion 504 in order to obtain an accurate FFR measurement, as explained above.

Pressure sensing portion 308 of elongate shaft 302 is located proximally of distal portion 310 and distal of proximal portion 306. Pressure sensing portion 308, as shown in FIG. 15, includes guide wire lumen 312 and a differential pressure sensor 316. Differential pressure sensor 316 includes an inner pressure sensing surface 318 that faces guide wire lumen 312 and an outer pressure sensing surface 319 that faces outside of shaft 302 into lumen 502 of vessel 500 when catheter is disposed in lumen 502. Differential pressure sensor 316 measures a differential pressure between the outside surface of shaft 302 and the guidewire lumen 312. Thus, measurement catheter 300 is disposed in vessel 500 with distal opening 311 disposed in distal lumen 508 and differential pressure sensor 316 disposed in proximal lumen 506. Lumen 312 is filled with blood 511 from distal opening 311 such that the blood 511 filling the lumen 312 contacts inner pressure sensing surface 318. Blood outside of measurement catheter 300 in proximal lumen 506 contacts outer pressure sensing surface 319. Thus, differential pressure sensor 316 measures the difference in pressure of blood (ΔP) outside of shaft 302 in proximal lumen 506 (i.e., Pₐ) and the pressure of the blood in lumen 312 (i.e. P_{d}). Thus, differential pressure sensor 316 measures differential pressure ΔP=Pₐ−P_{d}. In the embodiment shown, differential pressure sensor 316 does not directly measure Pₐ and P_{d}. Instead, differential pressure sensor 316 measures ΔP, as explained in more detail below.

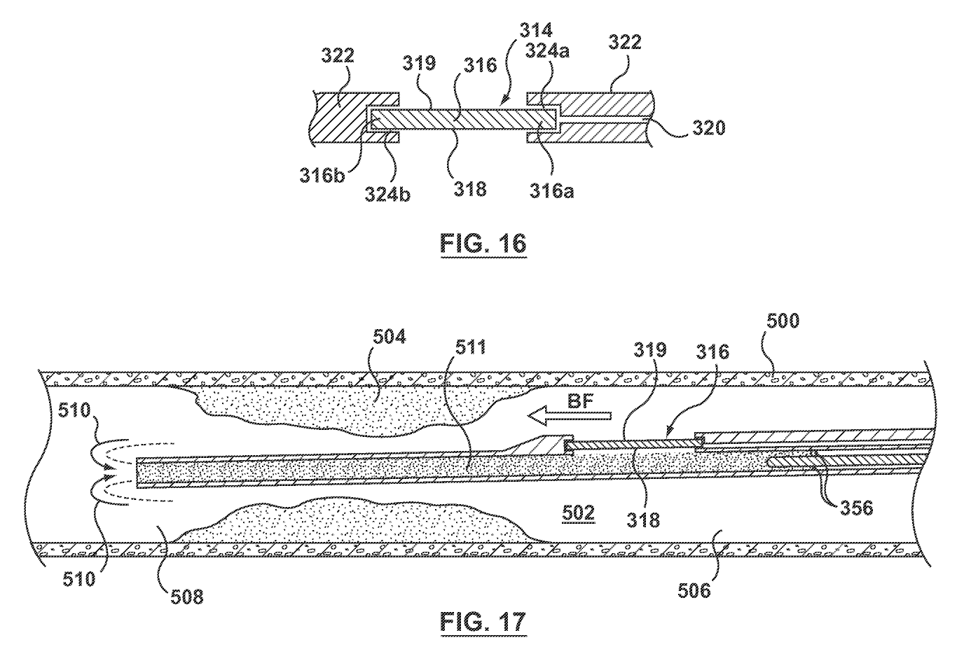

In the embodiment shown in FIG. 16, differential pressure sensor 316 is disposed in an opening 314 of a thickened portion 322 of elongate shaft 302. Differential pressure sensor 316 may be a piezo-resistive pressure sensor, a piezo-electric pressure sensor, a capacitive pressure sensor, an electromagnetic pressure sensor, an optical pressure sensor, and/or combinations thereof, or other pressure sensors that can measure blood pressure. Examples of commercially available pressure sensors suitable for used as differential pressure sensor 316 include, without limitation, a GE P160 sensor. As shown in FIG. 15, thickened portion 322 needs to accommodate differential pressure sensor 316. Accordingly, thickened portion 322 of elongate shaft 302 has an outer diameter OD₂ which is larger than the outer diameter OD₁ of distal portion 310 of elongate shaft 302. In one embodiment, outer diameter OD₂ of thickened portion 322 is in the range of 0.024 inch to 0.030 inch in order to accommodate differential pressure sensor 316. However, OD₂ may be a different size depending on the size of differential pressure sensor 116, thickness of elongate shaft 302, and other factors used to determine the diameter or profile of shafts.

In an embodiment, opening 314 is in communication with pressure sensor wire lumen 320 such that any communication wire(s) 321 from differential pressure sensor 316 may extend from opening 314 proximally through pressure sensor wire lumen 320, through a corresponding lumen in luer 330 exiting through proximal port (not shown) to a computing device 336 coupled to proximal end 323 of communication wire 321. Proximal end 323 of communication wire 321 may be coupled to computing device 336 via various communication pathways, including but not limited to one or more physical connections including electrical, optical, and/or fluid connections, a wireless connection, and/or combinations thereof. Accordingly, it is understood that additional components (e.g., cables, connectors, antennas, routers, switches, etc.) not illustrated in FIG. 15 may be included to facilitate communication between the proximal end 323 of communication wire 321 and computing device 336. In other embodiments, instead of a dedicated pressure sensor wire lumen 320, communication between differential pressure sensor 316 and computing device 336 may be accomplished wirelessly, or communication wires 321 may be incorporated into the wall of elongate shaft 302, or may be accomplished in various other ways.

In one embodiment shown in FIG. 16, opening 314 includes a first pocket 324a disposed and a first end 314a of opening 314 and a second pocket 324b at a second end 314a of opening 314. A first end 316a of differential pressure sensor 316 is disposed in first pocket 324a. A second end 316b of differential pressure sensor 316 is disposed in second pocket 324b. Accordingly, differential pressure sensor 316 is held in opening 314 of thickened portion 322 via pockets 324a, 324b. Ends 316a, 316b may be secured in pockets 324a, 324b, respectively, by adhesives, a press-fit, or various other ways.

In a method for measuring FFR using measurement catheter 300, a guide catheter (not shown) is advanced through the vasculature such that the guide catheter is disposed within the aorta with a distal end thereof disposed within the aorta at an ostium of the aorta adjacent the branch vessel 500 within which lesion 504 is located. Guidewire 150 is advanced intraluminally through the guide catheter, into vessel 500 within lumen 502 to the site of lesion 504, as shown and described with respect to FIG. 7. In the embodiment shown, guidewire 150 is advanced from proximal side 506 of lesion 504 to distal side 508 of lesion 504, which is also consistent with the direction of the blood flow BF, as indicated by the arrow BF in FIG. 7. In an embodiment, vessel 500 is a coronary artery, but vessel 500 may be other vessels in which it may be desirable to measure pressure, and in particular, to measure FFR.

Thereafter, measurement catheter 300 is tracked or advanced over indwelling guidewire 150 to the target site such that distal end 303 of elongate shaft 302 is positioned distal of lesion 504. Similar to the embodiment shown in FIG. 8, differential pressure sensor 316 is disposed proximal of lesion 504 such that the smaller profile distal portion 310 of elongate shaft 302 is disposed through lesion 504.

With measurement catheter 300 in place, guidewire 150 is retracted proximally until guidewire 150 is disposed proximal of differential pressure sensor 316, as described above with respect to FIG. 9. With guidewire 150 proximally retracted, blood 511 may enter distal opening 311 as indicated by arrows 510 and fill guidewire lumen 312 of elongate shaft 302 from a location at a distal end 154 of guidewire 150 to distal opening 311, as shown in FIG. 17. Further, although not shown in detail in FIG. 15-17, a seal 356, such as seal 156 shown in FIG. 11, may be provided along an inner surface of thickened portion 322 of elongated shaft 302 at approximately the location to where guidewire 150 is retracted such that blood 511 will not fill guidewire lumen 312 proximal of the seal.

With guidewire lumen 312 filled with blood 511, differential pressure sensor 316 measures the differential pressure ΔP between the outer pressure sensing surface 319 and the inner sensing surface 318. Outer pressure sensing surface 319 is in contact with the blood outside of shaft 302 on the proximal side 506 of lesion 504. Thus, the pressure of the blood acting on outer pressure sensing surface is indicative of Pₐ Inner pressure sensing surface 318 is in contact with blood 511 filling lumen 312 from distal opening 311 to distal end 154 of retracted guidewire 150. This pressure is indicative of pressure at the distal opening 311 because the blood filling lumen 312 transmits the pressure to inner pressure sensing surface 318. This blood pressure acting on inner pressure sensing surface 318 is the pressure on the distal side 508 of lesion 504, or P_{d}. Accordingly, the differential pressure measured ΔP by differential pressure sensor 316 is indicative of the proximal pressure minus the distal pressure, or Pₐ−P_{d}, which can be used to calculate FFR as described below. As explained above, differential pressure sensor 316 does not directly measure Pₐ or Pₐ. Instead, differential pressure sensor measures ΔP, i.e., the difference in pressure acting on the outer pressure sensing surface 319 and the inner pressure sensing surface 318.

In one embodiment, adenosine is administered either intracoronary at the site, bolus, or intravenously by continuous infusion for providing an accurate differential pressure measurement for an FFR value. A proximal pressure measurement Pₐ, which is taken in the aorta by an external AO pressure transducer associated with the guide catheter, and a simultaneous differential pressure measurement ΔP taken with differential pressure sensor 316 of measurement catheter 300 are then obtained to provide the FFR value, i.e., P_{d}/Pₐ, for the lesion. In particular, the following calculations are used to obtain the FFR value.

ΔP=Pₐ−Pₐ (1)

Rearranging the equation results in: P_{d}=Pₐ−ΔP (2)

Further, we know that: FFR=P_{d}/Pₐ (3)

Thus, substituting equation 2 into equation 3: FFR=(Pₐ−ΔP)/Pₐ (4)

The proximal pressure measurement Pₐ (taken using the guide catheter) and the differential pressure measurement ΔP are communicated to computing device 336. Computing device 336 may include such components as a CPU, a display device, an amplification and filtering device, an analog-to-digital converter, and various other components used to calculate and display FFR. Computing device 136 receives the proximal pressure measurement Pₐ and differential pressure measurement ΔP, processes them as shown in equation (4) above and known by those skilled in the art, and provides a continuous display of FFR measurement.

When the FFR measurement is completed, measurement catheter 300 may then be completely withdrawn from the patient or repositioned in vivo at another lesion and the process repeated.

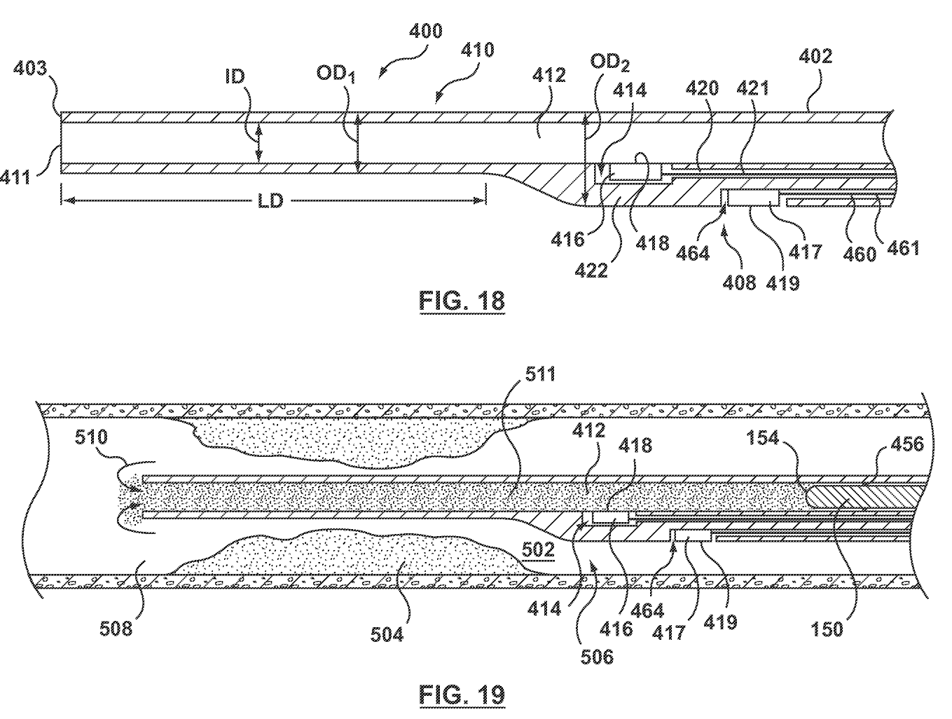

FIGS. 18-20 show a measurement catheter 400 and method of using measurement catheter 400 to calculate FFR in accordance with another embodiment hereof. Measurement catheter 400 is similar to measurement catheters 100 and 300 except that measurement catheter 400 includes two pressure sensors 416, 417 arranged such that pressure sensor 416 faces inward to measure a pressure of blood within a lumen of catheter 400 and pressure sensor 417 faces outward to measure a pressure of blood outside of catheter 400, as explained in more detail below. Further, while measurement catheter 400 is described below as similar to measurement catheter 100 in that an inner diameter ID of the distal portion 410 of measurement catheter 400 is sized approximately the same size as guidewire 150, distal portion 410 may instead be sized similar to distal portion 210 of measurement catheter 200 such that guidewire 150 need not be retracted.

Measurement catheter 400 includes an elongate shaft 402 having a proximal end (not shown) coupled to a handle or luer (not shown) and a distal end 403 having a distal opening 411. Elongate shaft 402 includes a proximal portion (not shown), an intermediate or pressure sensing portion 408, and a distal portion 410, as described in more detail below. Elongate shaft 402 may be formed of the materials described above with respect to elongate shaft 102. The proximal portion of catheter 400 is not shown in FIGS. 18-20, but can be the same as the proximal portions 110, 210, and 310 shown with respect to the other embodiments herein.

Elongate shaft 402 includes a guide wire lumen 412 extending therethrough. In the embodiment shown in FIGS. 18-20, guide wire lumen 412 extends through proximal portion (not shown), pressure sensing portion 408, and distal portion 410. However, instead of the over-the-wire configuration shown in FIGS. 18-20, catheter 400 may have a rapid exchange configuration wherein guide wire lumen 412 extends through distal portion 410 and intermediate portion 408, and guidewire 150 exits shaft 402 through a rapid exchange port (not shown) in the proximal portion (not shown). The proximal portion (not shown) may be similar to proximal portion 106 of catheter 100 shown in FIG. 3, including guidewire lumen 412 and a first pressure sensor wire lumen 420 extending therethrough. Further, a second pressure wire lumen 460, as described in more detail below, may be provided through the proximal portion (not shown). Further, although depicted as circular in cross-section, one or more lumen(s) of elongate shaft 302 may have any suitable cross-section including for example circular, elliptical, or crescent-shaped. First pressure sensing wire lumen 420 extends to pressure sensing portion 408 of elongate shaft 402 such that a wire 421 disposed therein may be coupled to first pressure sensor 416. Similarly, second pressure sensing wire lumen 460 extends to pressure sensing portion 408 such that a wire 461 disposed therein may be coupled to second pressure sensor 417. As further explained below, pressure sensor wire lumens 420, 460 may be eliminated in embodiments wherein signals from first and second pressure sensors 416, 417 are sent to the computing device (not shown) other than via wires 421, 461 in a dedicated pressure sensor wire lumens 420, 460, respectively, such as, but not limited to, wireless transmission or integration of wires 421, 461 into the wall of elongate shaft 402.

Distal portion 410 of elongate shaft 402 is configured to receive a guidewire 150 in guide wire lumen 412. As explained above with respect to the embodiment of FIGS. 1-11, in the embodiment of FIGS. 18-20, guidewire lumen 412 in distal portion 410 of shaft 402 is sized to receive guidewire 150. However, any additional cross-sectional size or profile of distal portion 410 is minimized such as to minimize the profile of distal portion 410 crossing lesion 504. Accordingly, in an embodiment for use with a 0.014 inch guidewire, distal portion 310 has an inner diameter ID of approximately 0.0155 inch and an outer diameter OD₁ of approximately 0.018 inch. However, these sizes are provided only as examples and may change depending on various factors such as, but not limited to, the size of the guidewire, the size of the pressure sensor, the particular vessel targeted, and various other factors. Further, if the profile of FIGS. 18-20 is used (similar to FIGS. 1-11), guidewire 150 will need to be retracted to fill lumen 412 such that the pressure measurement from first pressure sensor 416 described below may be taken. However, instead of the profile of FIGS. 1-11, distal portion 410 of measurement catheter 400 may instead have the profile of the embodiment of FIGS. 12-14 such that the guidewire 150 need not be refracted for lumen 412 to have sufficient fluid for a pressure measurement by first pressure sensor 416.

Further, as shown in FIG. 19, distal portion 410 is sized to extend from a proximal side 506 of lesion 504, through lesion 504, and to a distal side 508 of lesion 504 such that distal opening 411 is disposed on distal side 508 of lesion 504. Accordingly, in an embodiment, distal portion 310 has a length L_{D} in the range of 25-150 mm. However, length L_{D} may be any length suitable such that distal portion 410 may extend from proximal side 506 to distal side 508. Further, because distal portion 410 is configured to extend through lesion 504, the cross-sectional dimension or profile of distal portion 410 is minimized so as not to disrupt blood flow through lesion 504 in order to obtain an accurate FFR measurement, as explained above.

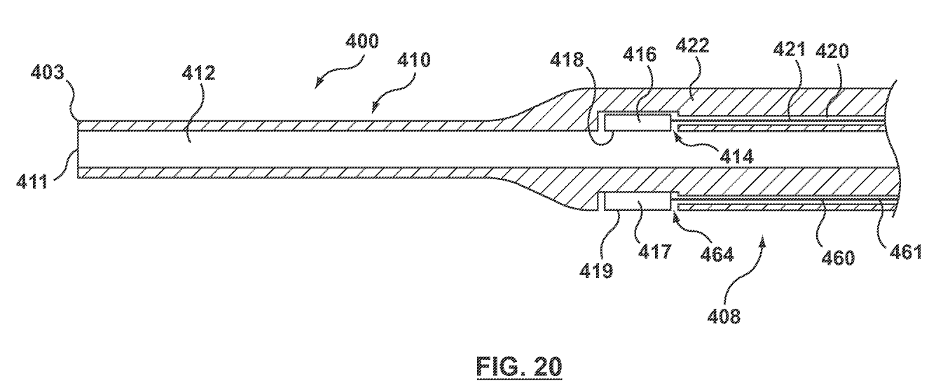

Pressure sensing portion 408 of elongate shaft 402 is located proximally of distal portion 410 and distal of the proximal portion (not shown). Pressure sensing portion 408, as shown in FIG. 18, includes guide wire lumen 412, first sensor 416, and second pressure sensor 417. In the embodiment shown in FIG. 18, first pressure sensor 416 and second pressure sensor are longitudinally offset from each other and circumferentially aligned with each other. Such an arrangement tends to minimize the outer diameter of shaft 402 at pressure sensing portion 408. However, other arrangements of first and second pressure sensors 416, 417 are also possible. For example, and not by way of limitation, FIG. 20 shows first and second pressure sensors 416, 417 longitudinally aligned and circumferentially offset by 180 degrees around the circumference of shaft 402. Other arrangements may also be used, such as, but not limited to: first and second pressure sensors 416, 417 being longitudinally aligned and circumferentially offset by 90 degrees or some other amount; first and second pressure sensors 416, 417 being longitudinally offset and circumferentially offset; and first and second pressure sensors being longitudinally aligned and circumferentially aligned such that first and second pressure sensors 416, 417 are “back-to-back”. In another example, first and second pressure sensors 416, 417 are longitudinally aligned and circumferentially aligned around the circumference of shaft 402.

First pressure sensor 416 includes an inner pressure sensing surface 418 that faces guide wire lumen 412. Second pressure sensor 417 includes an outer pressure sensing surface 419 that faces outside of shaft 402 into lumen 502 of vessel 500 when catheter 400 is disposed in lumen 502. Thus, first pressure sensor 416 measures pressure of blood 511 within lumen 412 when catheter 400 is in lumen 502, as described in more detail below. Second pressure sensor measures pressure of blood outside of shaft 402 in lumen 502 of vessel 500. Thus, measurement catheter 400 is disposed in vessel 500 with distal opening 411 disposed in distal lumen 508 and first and second pressure sensors 416, 417 in pressure sensing portion 408 disposed in proximal lumen 506. Lumen 412 is filled with blood 511 from distal opening 411 such that the blood 511 filling the lumen 412 contacts inner pressure sensing surface 418 of first pressure sensor. Blood outside of measurement catheter 400 in proximal lumen 506 contacts outer pressure sensing surface 419 of second pressure sensor 417. First pressure sensor 416 measures pressure of blood within lumen 512, which is blood pressure at distal opening 511. Thus, first pressure sensor 416 measures blood pressure in distal lumen 508, Second pressure sensor 417 measures pressure of blood outside of shaft 402 where outer pressure sensing surface 419 of second pressure sensor 417 is exposed to blood in proximal lumen 506. Thus, first pressure sensor 416 provides a measurement of blood pressure in distal lumen 508 (i.e., P_{d}) and second pressure sensor 417 provides a measurement of blood pressure in proximal lumen 506 (i.e., Pₐ). FFR can be calculated by the calculation FFR=P_{d}/Pₐ.

In the embodiment shown in FIG. 18, first pressure sensor 416 is disposed in a first pocket 414 of a thickened portion 422 of elongated shaft 402. Similarly, second pressure sensor 417 is disposed in a second pocket 464 in thickened portion 422 of elongate shaft 402. First and second pressure sensors 416, 417 may be piezo-resistive pressure sensors, piezo-electric pressure sensors, capacitive pressure sensors, electromagnetic pressure sensors, optical pressure sensors, and/or combinations thereof. As shown in FIG. 18, thickened portion 422 needs to accommodate first and second pressure sensors 416, 417. Accordingly, thickened portion 422 of elongate shaft 402 has an outer diameter OD₂ which is larger than the outer diameter OD₁ of distal portion 410 of elongate shaft 402. However, depending on the size of pressure sensors 416, 417, the OD₁ and OD₂ of the elongate shaft 102 could have the substantially the same diameter. In one embodiment, outer diameter OD₂ of thickened portion 422 is in the range of 0.018 inch-0.036 inch in order to accommodate first and second pressure sensors 416, 417. However, OD₂ may vary depending on the size of the pressure sensors, thickness of elongate shaft 402, and other factors used to determine the diameter or profile of shafts. First and second pockets 414, 464 may be as described in FIGS. 5 and 6 above, except that second pocket 464 faces an outside surface of elongate shaft 402 above or other configurations to accommodate first and second pressure sensors 416, 417.

In an embodiment, first pocket 414 is in communication with first pressure sensor wire lumen 420 such that any communication wire(s) 421 from first pressure sensor 416 may extend from first pocket 414 proximally through first pressure sensor wire lumen 420, through a corresponding lumen in a luer (not shown) exiting through proximal port (not shown) to a computing device (not shown) coupled to a proximal end (not shown) of communication wire 421, as described above with respect to other embodiments. Similarly, second pocket 464 is in communication with second pressure sensor wire lumen 460 such that any communication wire(s) 461 from second pressure sensor 417 may extend from second pocket 464 proximally through second pressure sensor wire lumen 460, through a corresponding lumen in a luer (not shown) exiting through proximal port (not shown) to a computing device (not shown) coupled to a proximal end (not shown) of second communication wire 461, as described above with respect to other embodiments. In other embodiments, instead of a dedicated pressure sensor wire lumens 420, 460, communication between first and second pressure sensors 416, 417 and the computing device may be accomplished wirelessly, or communication wires 421, 461 may be incorporated into the wall of elongate shaft 402.

In a method for measuring FFR using measurement catheter 400, a guide catheter (not shown) is advanced through the vasculature until a distal end thereof is disposed within the aorta proximal of an ostium of the branch vessel 500 within which lesion 504 is located. Guidewire 150 is advanced intraluminally through the guide catheter, into vessel 500 within lumen 502 to the site of lesion 504, as shown and described with respect to FIG. 7. In the embodiment shown, guidewire 150 is advanced from proximal side 506 of lesion 504 to distal side 508 of lesion 504, which is also consistent with the direction of the blood flow BF, as indicated by the arrow BF in FIG. 7. In an embodiment, vessel 500 is a coronary artery, but vessel 500 may be other vessels in which it may be desirable to measure pressure, and in particular, to measure FFR.

Thereafter, measurement catheter 400 is tracked or advanced over indwelling guidewire 150 to the target site such that distal end 403 of elongate shaft 402 is positioned distal of lesion 504. Similar to the embodiment shown in FIG. 8, first and second pressure sensors 416, 417 are disposed proximal of lesion 504 such that the smaller profile distal portion 410 of elongate shaft 402 is disposed through lesion 504.

With measurement catheter 400 in place, guidewire 150 is retracted proximally until guidewire 450 is disposed proximal of first pressure sensor 416, as described above with respect to FIG. 9. With guidewire 150 proximally refracted, blood 511 may enter distal opening 411 as indicated by arrows 510 and fill guidewire lumen 412 of elongate shaft 402 from a location at a distal end 154 of guidewire 150 to distal opening 411, as shown in FIG. 19. Further, although not shown in detail in FIGS. 18-20, a seal 456, such as seal 156 shown in FIG. 11, may be provided along an inner surface of thickened portion 422 of elongate shaft 402 at approximately the location to where guidewire 150 is retracted such that blood 511 will not fill guidewire lumen 412 proximal of the seal.

With guidewire lumen 412 filled with blood 511, inner pressure sensing surface 418 of first pressure sensor 416 measures the pressure of blood 511 within guidewire lumen 412. The pressure measured by first pressure sensor 416 is representative of the pressure of blood 511 at the distal side 508 of lesion 504 where the blood 511 enters distal opening 411 because the blood filling lumen 412 transmits the pressure to first pressure sensor 416. Accordingly, the pressure measured by first pressure sensor 116 is the distal pressure measurement, or P_{d}, used in calculating FFR. Similarly, outer pressure sensing surface 419 of second pressure sensor 417 is in contact with the blood 511 in lumen 502 on the proximal side 506 of lesion 504. Thus, second pressure sensor 417 measures pressure of blood outside of catheter 400 in proximal lumen 506, i.e., Pₐ.

In one embodiment, adenosine is administered either intracoronary at the site, bolus, or intravenously by continuous infusion for providing accurate pressure measurements for an FFR value. A proximal pressure measurement Pₐ, is taken by second pressure sensor 417 as described above, and a simultaneous distal pressure measurement P_{d} is taken by first pressure sensor 416, as described above. FFR can be calculated by the formula FFR=P_{d}/Pₐ.

The proximal pressure measurement Pₐ (taken using second pressure sensor 417) and the distal pressure measurement (taken using first pressure sensor 416) are communicated to the computing device (not shown). The computing device may include such components as a CPU, a display device, an amplification and filtering device, an analog-to-digital converter, and other components as needed. The computing device receives the distal pressure measurement P_{d} and the proximal pressure measurement Pₐ, and calculates FFR as shown in the equation above to provide a continuous display of FFR measurement.

When the FFR measurement is completed, measurement catheter 400 may then be completely withdrawn from the patient or repositioned in vivo at another lesion and the process repeated.

In embodiments hereof, an elongate tubular shaft or component and/or segments thereof may be formed of polymeric materials, non-exhaustive examples of which include polyethylene terephthalate (PET), polypropylene, polyethylene, polyether block amide copolymer (PEBA), polyamide, fluoropolymers, and/or combinations thereof, either laminated, blended or co-extruded. In other embodiments of an elongate tubular shaft or component in accordance herewith, a proximal segment thereof may be a hypotube of a medical grade stainless steel with outer and inner tubes of a distal segment thereof being formed from any of the polymeric materials listed above.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. For example, and not by way of limitation, measurement catheter 300 of FIGS. 15-17 may include an enlarged distal portion as shown in the embodiment of FIGS. 12-14 such that the guidewire does not need to be retracted in order to allow the lumen to fill with blood. All patents and publications discussed herein are incorporated by reference herein in their entirety.

What is claimed is:

1. A catheter comprising:
an elongate shaft including a proximal portion and a distal portion extending from the proximal portion to a distal opening at a distal end of the shaft, the elongate shaft defining a guidewire lumen, wherein the proximal portion has a first outer diameter and the distal portion has a second outer diameter smaller than the first outer diameter; and
a pressure sensor coupled to the proximal portion such that a pressure sensing surface of the pressure sensor faces the proximal guidewire lumen,
wherein the guidewire lumen is configured to receive a guidewire and to provide fluid communication between the pressure sensor and the distal opening, and
wherein when the catheter is tracked to a treatment site within the vasculature, the pressure sensor is disposed proximal the treatment site, the distal opening is disposed distal to the treatment site, and the guidewire lumen fills with blood such that the blood in the guidewire lumen contacts the pressure sensing surface of the pressure sensor such that the pressure sensor senses a pressure of the blood at the distal end of the shaft.

2. The catheter of claim 1, wherein the guidewire lumen is configured such that the guidewire is retracted proximally past the pressure sensor such that the guidewire lumen fills with blood to a distal end of the guidewire.

3. The catheter of claim 2, wherein the guidewire lumen is substantially the same cross-sectional size as the guidewire in the distal portion of the elongate shaft.

4. The catheter of claim 1, wherein the guidewire lumen is has a cross-sectional size sufficiently larger than the guidewire such that the guidewire lumen in the distal portion can fill with blood to the pressure sensor such that pressure sensor can sense the pressure of the blood at the distal end of the shaft with the guidewire located in the guidewire lumen in the distal portion of the shaft.

5. The catheter of claim 4, wherein the guidewire lumen in the distal portion is substantially pear-shaped such that with the guidewire is disposed in a wide portion of the guidewire lumen in the distal portion, a narrow portion of the guidewire lumen in the distal portion can fill up with blood such that the pressure sensor can sense the pressure of the blood at the distal end of the shaft with the guidewire located in the guidewire lumen in the distal portion.

6. The catheter of claim 5, further comprising a seal disposed proximal to the pressure sensor and between the proximal shaft and the guidewire disposed in the guidewire lumen in the proximal portion to prevent blood from flowing in the guidewire lumen proximal to the seal.

7. The catheter of claim 6, wherein the seal is disposed adjacent the pressure sensor.

8. The catheter of claim 1, further comprising a seal disposed proximal to the pressure sensor and between the proximal shaft and the guidewire disposed in the guidewire lumen in the proximal portion to prevent blood from flowing in the guidewire lumen proximal to the seal.

9. A catheter comprising:
an elongate shaft including a proximal portion and a distal portion extending from the proximal portion to a distal opening at a distal end of the shaft, the shaft defining a guidewire lumen, wherein the proximal portion has a first outer diameter and the distal portion has a second outer diameter smaller than the first outer diameter; and
a differential pressure sensor coupled to the proximal portion, wherein the differential pressure sensor includes an inner pressure sensing surface facing the guidewire lumen in the proximal portion and an outer pressure sensing surface facing outside of an outer surface of the elongate shaft,
wherein the guidewire lumen is configured to receive a guidewire and to provide fluid communication between the differential pressure sensor and the distal opening, and
wherein when the catheter is tracked to a treatment site within the vasculature, the differential pressure sensor is disposed proximal the treatment site, the distal opening is disposed distal to the treatment site, and the guidewire lumen fills with blood to the location of the differential pressure sensor such that the differential pressure sensor senses a pressure difference between blood outside of the elongate shaft adjacent the outer pressure sensing surface of the differential pressure sensor and blood at the distal end of the shaft sensed by the inner pressure sensing surface of the differential blood pressure via the blood filling the guidewire lumen.

10. The catheter of claim 9, wherein the guidewire lumen is configured such that the guidewire is retracted proximally past the differential pressure sensor such that the guidewire lumen fills with blood.

11. The catheter of claim 9, wherein the guidewire lumen in the distal portion is substantially the same size as the guidewire.

12. The catheter of claim 9, wherein the guidewire lumen in the distal portion has a cross-sectional size sufficiently larger than the guidewire such that the guidewire lumen can fill up with blood to the location of the differential pressure sensor to enable the inner pressure sensing surface to sense the pressure of the blood at the distal end of the shaft with the guidewire located in the guidewire lumen in the distal portion.

13. The catheter of claim 12, wherein the guidewire lumen in the distal portion is substantially pear-shaped such that with the guidewire disposed in a wide portion of the guidewire lumen in the distal portion, a narrow portion of the guidewire lumen in the distal portion can fill up with blood such that the inner pressure sensing surface can sense the pressure of the blood at the distal end of the shaft with the guidewire located in the guidewire lumen in the distal portion.

14. The catheter of claim 13, further comprising a seal disposed proximal to the differential pressure sensor and between the proximal shaft and the guidewire disposed in the guidewire lumen to prevent blood from flowing in the guidewire lumen proximal to the seal.

15. The catheter of claim 13, wherein the seal is disposed adjacent the differential pressure sensor.

16. The catheter of claim 9, further comprising a seal disposed proximal to the differential pressure sensor and between the proximal shaft and the guidewire disposed in the guidewire lumen to prevent blood from flowing in the guidewire lumen proximal to the seal.

17. A catheter comprising:
an elongate shaft including a proximal portion and a distal portion extending from the proximal portion to a distal opening at a distal end of the shaft, the elongate shaft defining a guidewire lumen, wherein the proximal portion has a first outer diameter and the distal portion has a second outer diameter smaller than the first outer diameter;
a first pressure sensor coupled to the proximal portion such that a first pressure sensing surface of the first pressure sensor faces the proximal guidewire lumen; and
a second pressure sensor coupled to the proximal portion such that a second pressure sensing surface of the second pressure sensor faces an outside surface of the shaft,
wherein the proximal guidewire lumen and distal guidewire lumen are configured to receive a guidewire and to provide fluid communication between the pressure sensor and the distal opening, and
wherein when the catheter is tracked to a treatment site within the vasculature, the pressure sensor is disposed proximal to the treatment site, the distal opening is disposed distal to the treatment site, the guidewire lumen fills with blood such that the blood is contact with the first pressure sensing surface and the first pressure sensor senses a pressure of the blood at the distal end of the shaft, and the second pressure sensing surface is in contact with blood outside of the elongate shaft proximal to the treatment site such that second pressure sensor senses a pressure of the blood outside of the elongate shaft proximal of the treatment site.

18. The catheter of claim 17, wherein the guidewire lumen in the distal portion is configured such that the guidewire is retracted proximally past the first pressure sensor such that the guidewire lumen fills with blood from the distal opining to a distal end of the guidewire.

19. The catheter of claim 18, wherein the guidewire lumen has a cross-sectional size which is substantially the same as a cross-sectional size of the guidewire.

20. The catheter of claim 17, wherein the guidewire lumen has a cross-sectional size sufficiently larger than a cross-sectional size of the guidewire such that the guidewire lumen can fill with blood from the distal opening to the first pressure sensor with the guidewire located in the guidewire lumen in the distal portion of the shaft.

21. The catheter of claim 20, wherein the distal guidewire lumen is substantially pear-shaped such that with the guidewire is disposed in a wide portion of the guidewire lumen in the distal portion of the elongate shaft, a narrow portion of the guidewire lumen in the distal portion can fill up with blood to such that the first pressure sensor can sense the pressure of the blood at the distal end of the shaft with the guidewire located in the guidewire lumen in the distal portion.
